(19) United States
(12) Patent Application Publication
(10) Pub. No.: US 2026/0174802 A1
(43) Pub. Date: Jun. 25, 2026

(54) COMPOSITION FOR PROMOTING DIFFERENTIATION OF STEM CELLS

(71) Applicants: ELPHIS CELL THERAPEUTICS, Seoul (KR); UNIVERSITY-INDUSTRY COOPERATION GROUP OF KYUNG HEE UNIVERSITY, Yongin-si, Gyeonggi-do (KR)

(72) Inventors: Hyunsook HONG, Seoul (KR); Dae Yeon HWANG, Goyang-si (KR); Jeong Seop PARK, Seoul (KR)

(73) Assignees: ELPHIS CELL THERAPEUTICS, Seoul (KR); UNIVERSITY-INDUSTRY COOPERATION GROUP OF KYUNG HEE UNIVERSITY, Yongin-si, Gyeonggi-do (KR)

(21) Appl. No.: 19/124,580

(22) PCT Filed: Jun. 9, 2023
(86) PCT No.: PCT/KR2023/007953

§ 371 Date: Apr. 25, 2025

(30) Foreign Application Priority Data
Oct. 26, 2022 (KR) 10-2022-0139281

(51) Int. Cl. A61K 35/28; A61K 35/32

(52) U.S. Cl. CPC A61K 35/28; A61K 35/32

(57) ABSTRACT

The present disclosure relates to a composition for promoting efficient osteogenic differentiation of adipose-derived stem cells. It has been identified in the present disclosure that by treating, with FGF2 and/or HGF, in an early stage of differentiation, ADSCs derived from an older donor having reduced differentiation efficiency, damaged paracrine signaling function recovers such that differentiation efficiency can be maximized, and thus the present disclosure can be used as an in vitro process technology for promoting the efficiency of stem cell differentiation prior to or at the time of adipose stem cell transplantation.

### TECHNICAL FIELD

The present disclosure relates to a composition for promoting differentiation of stem cells, and more particularly, to a composition for promoting efficient osteogenic differentiation of adipose-derived stem cells.

### BACKGROUND ART

A cell therapy product is defined as a pharmaceutical product used for the purposes of treatment, diagnosis, and prevention through a series of actions (more-than-minimal manipulation), such as expanding or selecting living autologous, allogeneic, or xenogeneic cells in vitro or changing the biological characteristics of cells by other methods to restore the function of cells and tissues. Among these, a stem cell therapy product refers to a cell therapy product that specifically uses stem cells, and currently, has been actively developed in representative application fields where recovery and regeneration of lost cells are essential, but not well achieved naturally, such as neurological diseases, heart diseases, lung diseases, liver diseases, and cancer. The stem cells are cells that may differentiate into various cells that constitute biological tissues, and are a general term for undifferentiated cells in the pre-differentiation stage that may be obtained from each tissue of the embryo, fetus, and adult. The stem cells differentiate into specific cells depending on a differentiation stimulus (environment), have a characteristic of self-renewing and proliferating (expanding) cells identical to themselves through cell division, unlike cells that have completed differentiation and stopped cell division, and may also differentiate into other cells by different environments or different differentiation stimuli to have plasticity in differentiation. The stem cells are broadly divided into pluripotent embryonic stem cells (ES cells), which are obtained from embryos and have the potential (totipotent) to differentiate into all cells, and multipotent adult stem cells obtained from each tissue. The inner cell mass of the blastocyte, which is an early stage of embryonic development, is a part that will eventually form the fetus, and embryonic stem cells formed from the inner cell mass are stem cells that theoretically have the potential to differentiate into cells of all tissues that constitute an organism. That is, the embryonic stem cells are undifferentiated cells that can proliferate indefinitely and may differentiate into all cells, and the adult stem cells are cells that have the differentiation potential into various cells. The adult stem cells may be obtained from various portions, including bone marrow, dental tissue, and peripheral blood, but in particular, adipose tissue is known to be a rich source of stem cells with various potentials. Adipose-derived stem cells (ADSCs), like other adult stem cells, are cells derived from mesenchymal tissue and can not only differentiate into various types of cells, such as adipocytes, fibroblasts, smooth muscle cells, endothelial cells, and preadipocytes, but also differentiate into epithelial, cartilage, nerve, fat, and muscle cells. In addition, it has been reported that the cell proliferation rate is fast, the adipose tissue that serves as a material can be incidentally extracted in large quantities during liposuction to be easily obtained, can be easily isolated by enzymes, and has low incidence of disease after transplantation.

Meanwhile, bones are maintained through the balance of bone formation by osteoblasts and osteoclasts (Alliston T. et al. Interfering with bone remodelling. Nature. 2002; 416:686-687.). The maintaining of the balance between osteoblasts and osteoclasts is an essential element for maintaining bone homeostasis. The disruption of the balance between osteoclasts and osteoblasts due to aging occurs when the osteoclastic capacity of osteoclasts exceeds the osteogenic capacity of osteoblasts to break homeostasis, which means that it is important to inhibit bone resorption by osteoclasts in a method for the prevention and treatment of bone disease due to aging (Tanaka, Y., et al., 2005). Excessive activity of osteoclasts that exceeds the activity of osteoblasts causes various bone diseases and is characterized by a decrease in bone mass and structural deterioration of the skeleton (Kim N. et al. Osteoclast differentiation independent of the TRANCE-RANK-TRAF6 axis. J Exp Med. 2005; 202:589-595.). As these bone diseases, intractable bone diseases include osteoporosis, non-union fractures, osteonecrosis, osteomalacia, bone defects, etc. The osteoporosis, also known as osteopenia, means a metabolic bone disease with a quantitative decrease in bone components as a main lesion in a significantly decreased condition in bone mass compared to normal people. In general, the pathology itself of osteoporosis often has asymptomatic or mild symptoms, but once a fracture occurs, it is generally difficult to be treated, and even with osteosynthesis, it is difficult to be recovered sufficiently.

Traditional treatments for bone diseases include autografting, allografting, artificial bone grafting, etc., but have problems such as causing complications such as infection, hematoma, and the like at the bone harvesting site (autografting), the possibility of disease transmission from donors (allografting), and the lack of fundamental bone formation (artificial bone grafting).

### DISCLOSURE

### Technical Problem

An object of the present disclosure is to provide a composition for promoting differentiation of stem cells.

Another object of the present disclosure is to provide a composition for inducing osteogenic differentiation.

Yet another object of the present disclosure is to provide a pharmaceutical composition for preventing or treating bone disease.

Still another object of the present disclosure is to provide a stem cell transplantation adjuvant.

Still another object of the present disclosure is to provide a method for enhancing osteogenic differentiation potential of stem cells.

Still another object of the present disclosure is to provide a use of FGF2 or HGF for differentiation of stem cells.

Still another object of the present disclosure is to provide a use of FGF2 or HGF for inducing osteogenic differentiation.

Still another object of the present disclosure is to provide a use of stem cells treated with FGF2 or HGF for preventing or treating bone disease.

Still another object of the present disclosure is to provide a method for treating bone disease.

### Technical Solution

In order to achieve the objects, an aspect of the present disclosure provides a composition for promoting differentiation of stem cells including a fibroblast growth factor (FGF) 2 or a hepatocyte growth factor (HGF) as an active ingredient.

Another aspect of the present disclosure provides a composition for inducing osteogenic differentiation including FGF2 or HGF as an active ingredient.

Yet another aspect of the present disclosure provides a pharmaceutical composition for preventing or treating bone disease including FGF2 or HGF as an active ingredient.

Still another aspect of the present disclosure provides a stem cell transplantation adjuvant including FGF2 or HGF.

Still another aspect of the present disclosure provides a method for enhancing osteogenic differentiation potential of stem cells.

Still another aspect of the present disclosure provides a use of FGF2 or HGF for differentiation of stem cells.

Still another aspect of the present disclosure provides a use of FGF2 or HGF for inducing osteogenic differentiation.

Still another aspect of the present disclosure provides a use of stem cells treated with FGF2 or HGF for preventing or treating bone disease.

Still another aspect of the present disclosure provides a method for treating bone disease including transplanting FGF2 or HGF into a subject suffering from bone disease.

Still another aspect of the present disclosure provides a method for treating bone disease including transplanting stem cells treated with FGF2 or HGF into a subject suffering from bone disease.

### Advantageous Effects

According to the present disclosure, it was confirmed that ADSCs derived from elderly donors have reduced differentiation efficiency due to damaged secretion capacity of growth factors, and thus fail to form/generate bone even in response to osteogenesis-inducing stimulation, and exhibit damaged paracrine potential. Accordingly, it has been found that differentiation efficiency can be maximized by treating FGF2 and/or HGF in an early stage of differentiation to enhance the activity of stem cells, and thus the present disclosure can be used as an in vitro process technology for promoting the efficiency of differentiation of stem cells prior to or at the time of adipose stem cell transplantation.

### DESCRIPTION OF DRAWINGS

FIG. 1 is a diagram confirming the osteogenic/paracrine potential according to the age of a subject (donor) from which ADSCs are isolated:

FIG. 1A: Cell morphology of ADSC-Y and ADSC-E;

FIG. 1B: Cell doubling time;

FIG. 1C: Experimental schematic diagram for comparative analysis of osteogenesis of ADSC-Y and ADSC-E;

FIG. 1D: Alizarin Red S staining images of ADSC-Y and ADSC-E after osteogenesis induction for 20 days;

FIG. 1E: Quantitative graph of Alizarin Red S staining images;

FIG. 1F: Western blot analysis result of osteogenic markers after 0, 1, 3, and 6 days (D0, D1, D3, and D6) of osteogenesis induction;

FIG. 1G: Quantitative graph of Western blot analysis result of Runx-1 after 0, 1, 3, and 6 days of osteogenesis induction;

FIG. 1H: Quantitative graph of Western blot analysis result of ALP after 0, 1, 3, and 6 days of osteogenesis induction;

FIG. 1I: BMP-2 secretion levels of ADSC-Y and ADSC-E analyzed by ELISA;

FIG. 1J: VEGF secretion levels of ADSC-Y and ADSC-E analyzed by ELISA;

FIG. 1K: TGF-β1 secretion levels of ADSC-Y and ADSC-E analyzed by ELISA;

FIG. 1L: HGF secretion levels of ADSC-Y and ADSC-E analyzed by ELISA; and

FIG. 1M: Western blot analysis result and quantification graph of FGF-2 protein levels in ADSC-Y and ADSC-E.

FIG. 2 is a diagram analyzing expression patterns of osteogenic factors of ADSC-Y and ADSC-E during osteogenesis induction:

FIG. 2A: BMP-2 secretion levels of ADSC-Y and ADSC-E analyzed by ELISA after 0, 1, 3, and 6 days of osteogenesis induction;

FIG. 2B: TGF-β1 secretion levels of ADSC-Y and ADSC-E analyzed by ELISA after 0, 1, 3, and 6 days of osteogenesis induction;

FIG. 2C: VEGF secretion levels of ADSC-Y and ADSC-E analyzed by ELISA after 0, 1, 3, and 6 days of osteogenesis induction;

FIG. 2D: HGF secretion levels of ADSC-Y and ADSC-E analyzed by ELISA after 0, 1, 3, and 6 days of osteogenesis induction;

FIGS. 2E to 2G: Western blot analysis results and quantification graphs of P-Met and c-Met in ADSC-Y and ADSC-E after 0, 1, 3, and 6 days of osteogenesis induction; and

FIGS. 2H to 2J: Western blot analysis results and quantification graphs of FGFR2 and FGF2 in ADSC-Y and ADSC-E after 0, 1, 3, and 6 days of osteogenesis induction.

FIG. 3 is a diagram confirming an osteogenic function improvement effect of ADSC-E by FGF2 and/or HGF during osteogenesis induction:

FIG. 3A: Schematic diagram of an experiment for inducing osteogenesis and treating FGF2 and/or HGF; and

FIGS. 3B and 3C: Alizarin Red S staining images and quantification graphs of ADSC-E under each condition.

FIG. 4 is a diagram confirming an early expression regulation effect of osteogenic markers in ADSC-E by FGF2 and/or HGF during osteogenesis induction:

FIG. 4A: Schematic diagram of an experimental process of treating ADSC-E with FGF2 and/or HGF during osteogenesis induction and performing Western blot and ELISA analyses on days 1, 3, and 6;

FIGS. 4B to 4F: Protein expression levels of FGFR2, Runx-2, Osterix, and ALP in ADSC-E confirmed by Western blot analysis;

FIG. 4G: BMP-2 secretion level confirmed by ELISA; and

FIG. 4H: VEGF secretion level confirmed by ELISA.

FIG. 5 is a diagram confirming a bone formation potential improvement effect of ADSC by FGF2 and/or HGF in vivo:

FIG. 5A: Schematic diagram of an experiment for transplanting ADSC-E primed with FGF2 and/or HGF into a mouse;

FIGS. 5B and 5C: H&E staining results and quantification graphs thereof for transplanted cells and bone complexes; and

FIGS. 5D and 5E: Immunohistochemically stained human osteocalcin and quantification graph thereof.

### BEST MODE

Hereinafter, embodiments of the present disclosure will be described in detail with reference to the accompanying drawings. However, the following embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

In addition, terminologies used herein are terminologies used to properly express preferred embodiments of the present disclosure, which may vary according to a user, an operator's intention, or customs in the art to which the present disclosure pertains. Therefore, these terminologies used herein will be defined based on the contents throughout the specification. Throughout this specification, unless explicitly described to the contrary, when a certain part “comprises” a certain component, it will be understood to imply the inclusion of stated elements, not the exclusion of any other elements.

All technical terms used in the present disclosure, unless otherwise defined, are used as the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

Throughout this specification, ‘%’ used to indicate the concentration of a specific material is solid/solid (w/w) %, solid/liquid (w/v) %, and liquid/liquid (v/v) %, unless otherwise stated.

In an aspect, the present disclosure provides a composition for promoting differentiation of stem cells including a fibroblast growth factor (FGF) 2 or a hepatocyte growth factor (HGF) as an active ingredient.

In one embodiment, the composition of the present disclosure may include FGF2 and HGF together.

In one embodiment, the composition of the present disclosure may include FGF2 at a concentration of 0.5 to 10 ng/mL and HGF at a concentration of 5 to 100 ng/ml, most preferably FGF2 at a concentration of 5 ng/ml and HGF at a concentration of 50 ng/mL.

In one embodiment, the stem cells may be adult stem cells, and the adult stem cells may be derived from at least one selected from the group consisting of bone marrow, blood, brain, skin, fat, umbilical cord blood and umbilical cord Wharton's jelly, and most preferably adipose-derived stem cells (ADSCs).

In one embodiment, the composition of the present disclosure may promote differentiation of stem cells into osteocytes.

In one embodiment, the composition of the present disclosure may promote/enhance/stimulate differentiation into osteoblasts under osteogenic induction conditions (osteogenic differentiation media), in which the osteogenic induction conditions may be replaced with osteogenesis differentiation media from general culture media.

In one embodiment, the composition of the present disclosure may promote differentiation of adipose-derived stem cells (ADSCs) into osteoblasts.

In one embodiment, the stem cells may be adipose-derived stem cells (ADSC-E) derived from elderly donors aged 50 to 80 years, and the ADSC-E may have damaged paracrine potential and osteogenic function.

In one embodiment, the composition of the present disclosure may increase the osteogenic function of ADSC-E.

In one embodiment, the composition of the present disclosure may increase the bone formation/generation function of ADSC-E, thereby promoting differentiation into osteoblasts.

In one embodiment, the ADSC-E may have decreased expression of growth factors Runx-2 and ALP, decreased secretion of paracrine factors BMP-2, VEGF, TGF-Beta1, and HGF, and decreased expression of C-Met phosphorylation, FGF2, and FGF2R, compared to adipose-derived stem cells derived from young donors (ADSC-Y) aged 20 to 29 years.

In one embodiment, the composition of the present disclosure may increase the early expression of an osteogenic marker, in which the marker may be FGFR2, Runx-2, Osterix or ALP.

In one embodiment, the composition of the present disclosure may promote secretion of factors related to revascularization and osteogenesis, in which the factor may be BMP-2 or VEGF.

In one embodiment, the composition of the present disclosure may increase the expression of osteocalcin, a bone formation/generation marker.

In an embodiment, the composition of the present disclosure may be a medium composition.

In an aspect, the present disclosure provides a composition for inducing osteogenic differentiation including FGF2 or HGF as an active ingredient.

In one embodiment, the composition of the present disclosure may increase a differentiation-inducing effect when inducing osteocyte differentiation.

In one embodiment, the osteocytes may be osteoblasts.

In the present disclosure, the composition of the present disclosure including FGF2 or HGF as the active ingredient may be mixed with a cell therapy product for treatment and injected into the body, thereby enhancing an in vivo effect of the cell therapy product, and may also be used as a method of transplanting a cell therapy product with an increased function into the body after treating a stem cell itself with the composition.

In an aspect, the present disclosure provides a pharmaceutical composition for preventing or treating bone disease including FGF2 or HGF as an active ingredient.

In one embodiment, the bone disease may be one or more selected from the group consisting of arthritis, bone loss disease, osteoporosis, osteopenia, osteolytic metastasis, senile kyphosis and Paget disease. The arthritis may be synovitis, rheumatoid arthritis (RA), juvenile rheumatoid arthritis, osteoarthritis (OA), gout, pseudogout, spondyloarthritis (SpA), psoriatic arthritis, ankylosing spondylitis, septic arthritis, arthritis, juvenile idiopathic arthritis, blunt trauma, joint replacement or Still disease.

In one embodiment, the composition of the present disclosure may include FGF2 and HGF together with stem cells.

The pharmaceutical composition of the present disclosure may further include a known bone disease therapy product in addition to FGF2 and/or HGF as the active ingredient, and may be used in combination with other known treatments for the treatment of these diseases.

As used herein, the term “prevention” means all actions that inhibit or delay the occurrence, spread, and recurrence of bone diseases by administration of the pharmaceutical composition according to the present disclosure. The term “treatment” means all actions that improve or beneficially change the symptoms of bone diseases by administering the composition of the present disclosure. Those skilled in the art to which the present disclosure pertains will be able to determine the degree of improvement, enhancement and treatment by knowing the exact criteria of disease for which the composition of the present disclosure is effective by referring to data presented by the Korean Academy of Medical Sciences, etc.

As used herein, the term “therapeutically effective amount” used in combination with the active ingredients means an amount effective to prevent or treat bone disease, and the therapeutically effective amount of the composition of the present disclosure may vary depending on several factors, such as a method of administration, a target site, the condition of a patient. Accordingly, when used in the human body, a dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective amount. As used herein, the term “pharmaceutically effective amount” refers to an amount enough to treat the disease at a reasonable benefit/risk ratio applicable to medical treatment and not to cause side effects. The effective dose level may be determined according to factors including the health condition of a patient, the cause and severity of bone disease, the activity of a drug, the sensitivity to a drug, a method of administration, a time of administration, a route of administration, an excretion rate, duration of treatment, and combined or simultaneously used drugs, and other factors well-known in the medical field. The composition of the present disclosure may be administered as an individual therapy product or in combination with other therapy products, and may be administered sequentially or simultaneously with conventional therapy products, and may be administered singly or multiply. It is important to administer an amount capable of obtaining a maximum effect with a minimal amount without side effects by considering all the factors, which may be easily determined by those skilled in the art.

The pharmaceutical composition of the present disclosure may include carriers, diluents, excipients, or a combination of two or more thereof, which are commonly used in biological agents. As used herein, the term “pharmaceutically acceptable” means exhibiting non-toxic properties to cells or humans exposed to the composition. The carrier is not particularly limited as long as the carrier is suitable for in vivo delivery of the composition, and may be used by mixing, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

In one embodiment, the pharmaceutical composition may be one or more formulations selected from the group including oral formulations, external formulations, suppositories, sterile injection solutions and sprays, and more preferably oral or injectable formulations.

As used herein, the term “administration” means providing a predetermined substance to a subject or patient by any suitable method, and the pharmaceutical composition may be administered parenterally (e.g., intravenously, subcutaneously, intraperitoneally or topically) or orally according to a desired method. The dose range may vary depending on the body weight, age, sex, and health condition of a patient, a diet, an administration time, an administration method, an excretion rate, the severity of a disease, etc. Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present disclosure may also be administered by any device capable of transferring an active substance to a target cell. Preferred administration methods and formulations are intravenous injections, subcutaneous injections, intradermal injections, intramuscular injections, drop injections, etc. The injections may be prepared by using aqueous solvents such as a physiological saline solution and a Ringer's solution, and non-aqueous solvents such as vegetable oils, higher fatty acid esters (e.g., ethyl oleate), and alcohols (e.g., ethanol, benzyl alcohol, propylene glycol, or glycerin). The injections may include pharmaceutical carriers, such as a stabilizer for the prevention of degeneration (e.g., ascorbic acid, sodium hydrogen sulfite, sodium pyrosulfite, BHA, tocopherol, EDTA, etc.), an emulsifier, a buffer for pH control, and a preservative to inhibit microbial growth (e.g., phenyl mercury nitrate, thimerosal, benzalkonium chloride, phenol, cresol, benzyl alcohol, etc.).

As used herein, the term “subject” refers to all animals including monkeys, cows, horses, sheep, pigs, chickens, turkeys, quails, cats, dogs, mice, rats, rabbits or guinea pigs including humans who have developed or may develop the bone disease, and the pharmaceutical composition of the present disclosure may be administered to a subject to effectively prevent or treat the diseases. The pharmaceutical composition of the present disclosure may be administered in combination with existing therapeutic agents.

The pharmaceutical composition of the present disclosure may further include a pharmaceutically acceptable additive. At this time, the pharmaceutically acceptable additive may be used with starch, gelatinized starch, microcrystalline cellulose, milk sugar, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present disclosure is preferably included in an amount of 0.1 part by weight to 90 parts by weight based on the composition, but is not limited thereto.

In an aspect, the present disclosure provides a stem cell transplantation adjuvant including FGF2 or HGF.

In one embodiment, the transplantation adjuvant may be administered simultaneously or concurrently with the transplantation of stem cells, but is more preferably administered simultaneously or together, and may promote/enhance or promote osteogenic differentiation of the transplanted stem cells.

In one aspect, the present disclosure relates to a method for enhancing osteogenic differentiation potential of stem cells, including (a) isolating adipose-derived adult stem cells extracted from adipocytes isolated from a subject; and (b) treating the stem cells with FGF2 or HGF.

In one embodiment, step (b) may be priming the stem cells by replacing the medium with a differentiation medium containing FGF2 or HGF.

In one embodiment, FGF2 or HGF may be treated within 7 days after differentiation induction, and may be treated during initial 3 or 6 days.

In one embodiment, the method may be a method for enhancing the differentiation potential of adipose-derived stem cells into osteoblasts.

The term “priming” as used herein refers to a phenomenon in which the reactivity (activity) of stem cells is enhanced to promote the therapeutic efficacy of the stem cells.

In one aspect, the present disclosure relates to a method for producing a cell therapy product with enhanced osteogenic capacity, including priming adipose-derived adult stem cells isolated from a subject by treating FGF2 and/or HGF.

In one embodiment, the cell therapy product may be an autologous cell therapy product.

In one embodiment, the subject may be an elderly person.

In one embodiment, the cell therapy product may be primed in vitro with stem cells isolated from a patient through apheresis, and then injected again into the patient.

In an aspect, the present disclosure provides a use of FGF2 or HGF for differentiation of stem cells.

In an aspect, the present disclosure provides a use of FGF2 or HGF for inducing osteogenic differentiation.

In an aspect, the present disclosure provides a use of stem cells treated with FGF2 or HGF for preventing or treating bone disease.

In an aspect, the present disclosure provides a method for treating bone disease including transplanting FGF2 or HGF into a subject suffering from bone disease.

In an aspect, the present disclosure provides a method for treating bone disease including transplanting stem cells treated with FGF2 or HGF into a subject suffering from bone disease.

### [Modes]

Hereinafter, the present disclosure will be described in more detail with reference to the following Examples. However, the following Examples are only intended to embody the contents of the present disclosure, and the present disclosure is not limited thereto.

### Example 1. Isolation and Culture of Young ADSCs and Elderly ADSCs

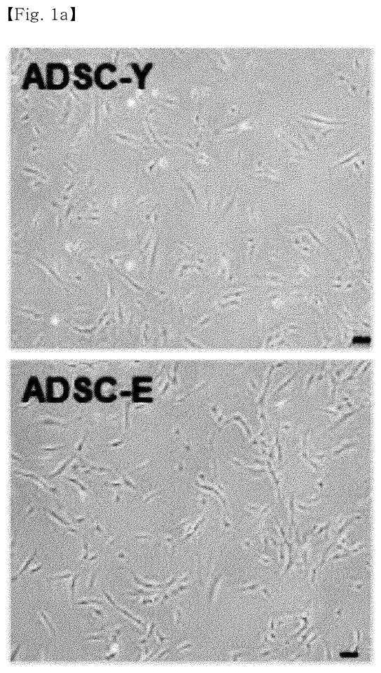

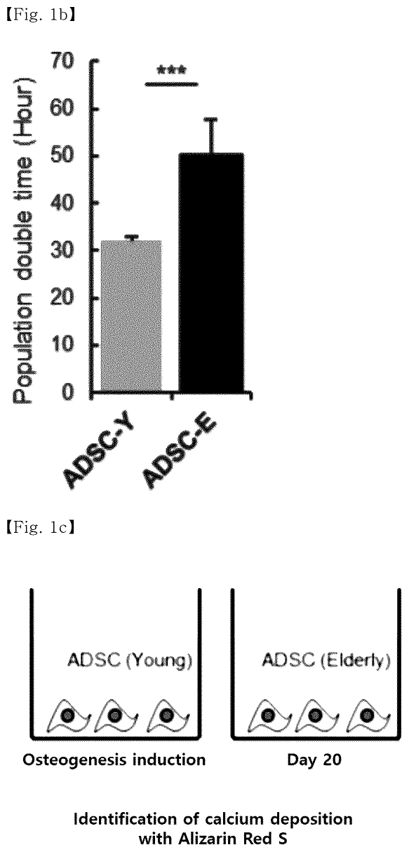

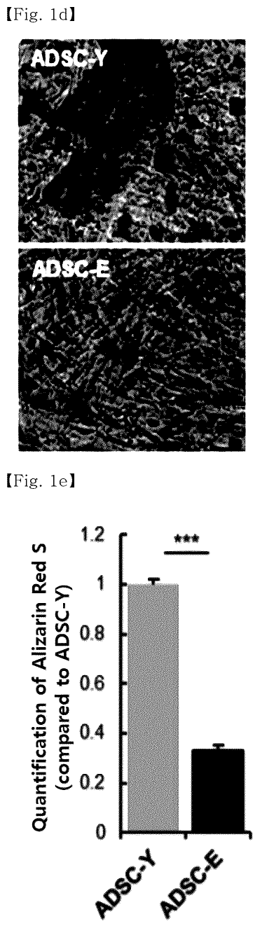

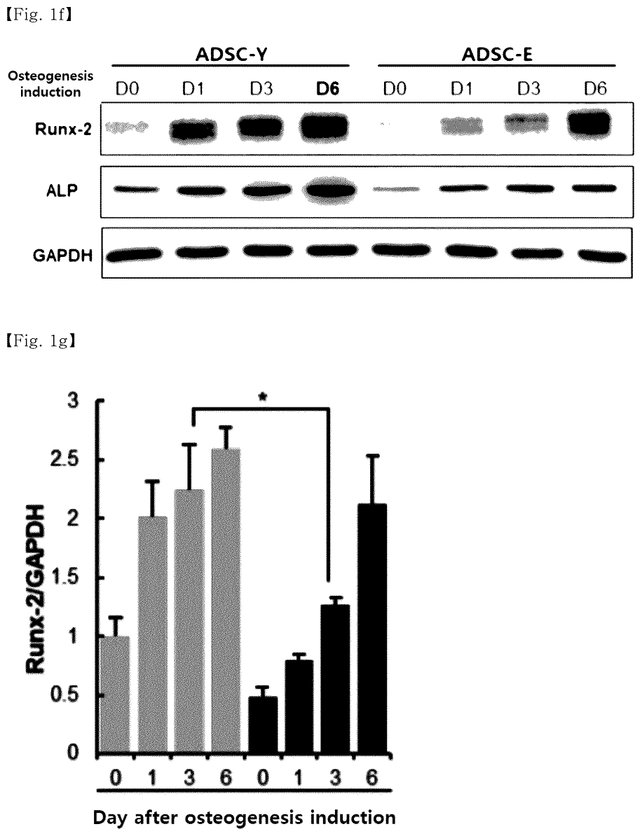

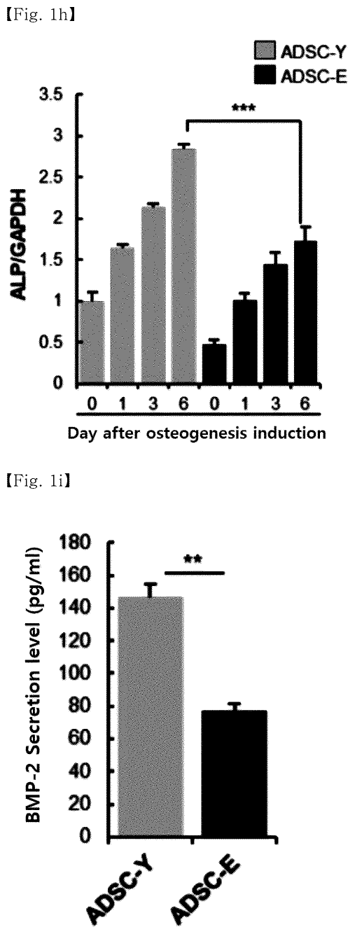

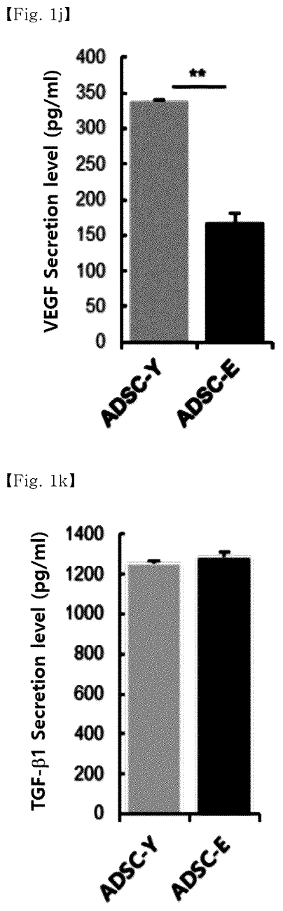

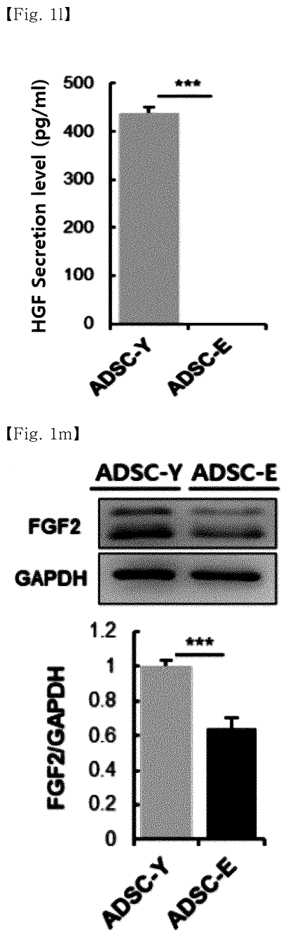

To compare and analyze the cellular functions of young ADSCs (ADSC-Y) and elderly ADSCs (ADSC-E), ADSCs were isolated and cultured from young and elderly donors, respectively. Specifically, elderly adipose tissues were collected from donors aged 50 to 70 years who gave written consent at the Kyung Hee University Hospital [Seoul, Korea; (IRB #2016-12-022, donor: 8, 2021-01-011, donor: 20)], washed with PBS (Welgene, Daegu, Korea) containing 5% penicillin/streptomycin, and enzymatically decomposed with 1% collagenase I for 1 hour at 37° C. The enzymatic reaction was stopped by adding an equal volume of FBS, and after centrifugation, the stromal vascular fraction was filtered through a cell strainer (70 μm, Corning, NY, USA) to remove debris. Thereafter, ADSC pellets were obtained by centrifugation at 1500 rpm for 5 minutes at 4° C. Adipose-derived stem cells (ADSCs) were resuspended in a-MEM containing 10% FBS, 1% penicillin, and streptomycin. ADSCs isolated from healthy donors aged 20 to 29 years were purchased from ScienCell Research Laboratories (Carlsbad, CA). All ADSCs were cultured in a 5% CO₂ incubator at 37° C., and the culture medium was replaced every other day. Thereafter, in the experiment, ADSCs of passages 3 to 5 were used. As a result of comparing the cell morphology and doubling time of the isolated young ADSCs (ADSC-Y) and elderly ADSCs (ADSC-E), almost no difference in cell morphology was observed (FIG. 1A), but ADSC-Y proliferated every 30 hours, whereas ADSC-E proliferated every 50 hours (FIG. 1B).

### Example 2. Analysis of Osteogenic Differentiation by Osteogenesis Induction

To evaluate the osteogenic function of young ADSCs and elderly ADSCs, the ADSCs were cultured in an osteogenic induction medium for 20 days, and then Alizarin Red S staining analysis was performed to confirm calcium deposition. Specifically, ADSC-Y and ADSC-E were dispensed at 5×10⁴ cells/well in a 6-well plate, respectively, and then, when the cell density reached 80 to 90%, the culture medium was replaced with Stempro osteogenesis differentiation media (Gibco, Grand Island, NY, USA) and cultured for 20 days to induce osteogenesis. On day 20 of osteogenesis induction, the cells were fixed with 3.7% formaldehyde (Sigma-Aldrich, ST. Louis, MO, USA) and stained with 2% Alizarin red S solution (Sigma-Aldrich, ST. Louis, MO, USA) for 10 minutes to visualize calcium deposition. Thereafter, Alizarin red S was eluted with a 10% cetylpyridinium chloride solution (Sigma-Aldrich, ST. Louis, MO, USA), and calcium deposition was quantified by the absorbance value at 560 nm (Molecular Devices, Sunnyvale, CA, USA).

As a result, ADSC-Y was able to differentiate into osteoblasts under conditions of high calcium deposition, whereas ADSC-E showed little differentiation into osteoblasts even under the same osteogenesis-inducing conditions (FIGS. 1C to 1E).

### Example 3. Analysis of Production of Osteogenesis-Related Growth Factors According to Osteogenesis Induction

The expression levels of transcription regulators in ADSC-Y and ADSC-E under osteogenesis induction conditions were confirmed by Western blot analysis. Specifically, ADSC-Y and ADSC-E after 0, 1, 3, and 6 days of osteogenesis induction in Example 2 were washed with PBS, lysed with 1× lysis buffer (Cell Signaling Technology, Danvers, MA, USA), and centrifuged at 12,000 rpm at 4° C. for 20 minutes to collect the supernatant. The protein concentration in the supernatant was determined by bicinchoninic acid (BCA) analysis (Thermo Fisher, Rockford, IL, USA) and electrophoresed using SDS-PAGE. Thereafter, the supernatant was transferred to a nitrocellulose membrane, blocked with 5% skim milk, and incubated with primary antibodies against Runx-2 (Cell Signaling Technology, Danvers, MA, USA), alkaline phosphatase (ALP) (Abcam, Cambridge, UK), and glyceraldehyde 3-phosphate dehydrogenase (GAPDH) (Abcam, Cambridge, UK), and then reacted with an anti-IgG horseradish peroxidase (HRP)-conjugated secondary antibody (Bio-rad, Hercules, CA, USA). The blot was developed by adding ECL (Dogen Bio, Seoul, Korea) and chemiluminescence was visualized with an Amersham imager 600 (GE Healthcare, Buckinghamshire, UK). The expression level of each protein was quantified using the ImageJ program (Version 1.53e, National Institutes of Health, Bethesda, Maryland, USA).

As a result, it was found that osteogenic cell differentiation was performed through the activation of Runx-2, one of the major transcription regulators of early osteogenesis, and the expression of Runx-2 was lower in ADSC-E than in ADSC-Y in the early stage of osteogenesis induction (FIGS. 1F and 1G). In addition, it was found that the expression of ALP was higher in ADSC-Y (FIGS. 1F and 1H). It was confirmed that the loss of osteogenic function of ADSC-E was associated with a decrease in the expression of the growth factors.

### Example 4. Comparison of Paracrine Factor Production in Young ADSCs And Elderly ADSCs

Since activation of Runx-2 was associated with signaling molecules such as TGF-Beta, FGF, and BMP-2, to confirm the paracrine potential of ADSC-Y and ADSC-E, the secretion of osteogenesis-enhancing growth factors including BMP-2, VEGF, TGF-Beta1, and HGF in ADSC-Y and ADSC-E was evaluated by ELISA. As a result, the levels of BMP-2 and VEGF were significantly higher in ADSC-Y than in ADSC-E (FIGS. 1I and 1J), and contrary to expectations, the production of TGF-Beta1 was not affected by age and osteogenic capacity in ADSC (FIG. 1K). In addition, there was a significant difference in the secretion of hepatocyte growth factor (HGF) in ADSC-Y and ADSC-E, which indicated that the secretion of HGF was deeply associated with the osteogenic function of ADSC. In addition, it was confirmed that the level of fibroblast growth factor-2 (FGF-2), a representative factor for promoting osteogenesis, was significantly lower in ADSC-E than in ADSC-Y (FIG. 1M).

Through these results, it can be seen that aging decreases cell repopulation rate and differentiation function, which is caused by a decrease in paracrine factors.

### Example 5. Analysis of Paracrine Factor Production According to Osteogenesis Induction

Since ADSCs with deficient osteogenic function as described above exhibited damaged paracrine potential in response to osteogenic stimulation, the kinetics of osteogenesis-related paracrine factors during osteogenesis induction were investigated in ADSC-Y and ADSC-E, respectively, to confirm a correlation between the osteogenic function and the paracrine factors. Specifically, ADSC-Y and ADSC-E were cultured in an osteogenic medium, respectively, and a conditioned medium was collected after 0, 1, 3, and 6 days of osteogenesis induction, and the levels of BMP-2, TGF-β1, VEGF, and HGF were analyzed by ELISA.

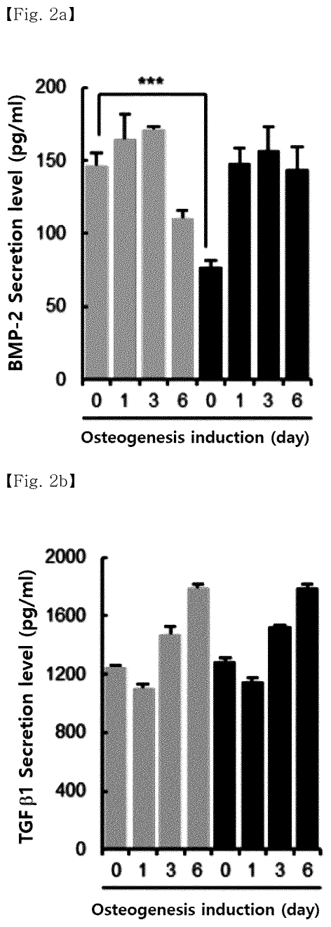

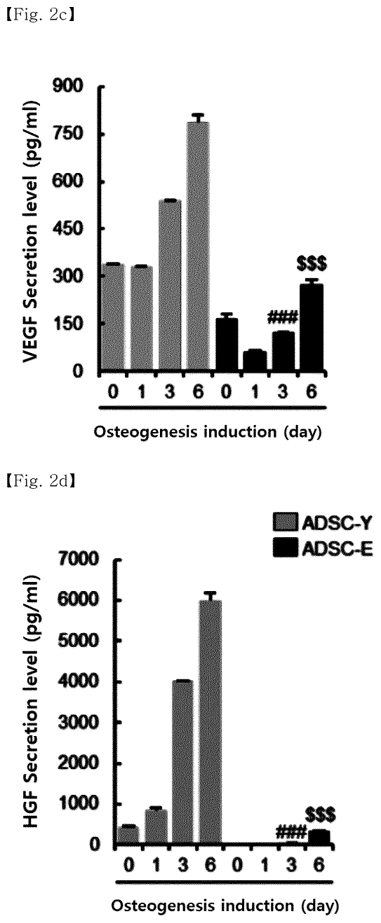

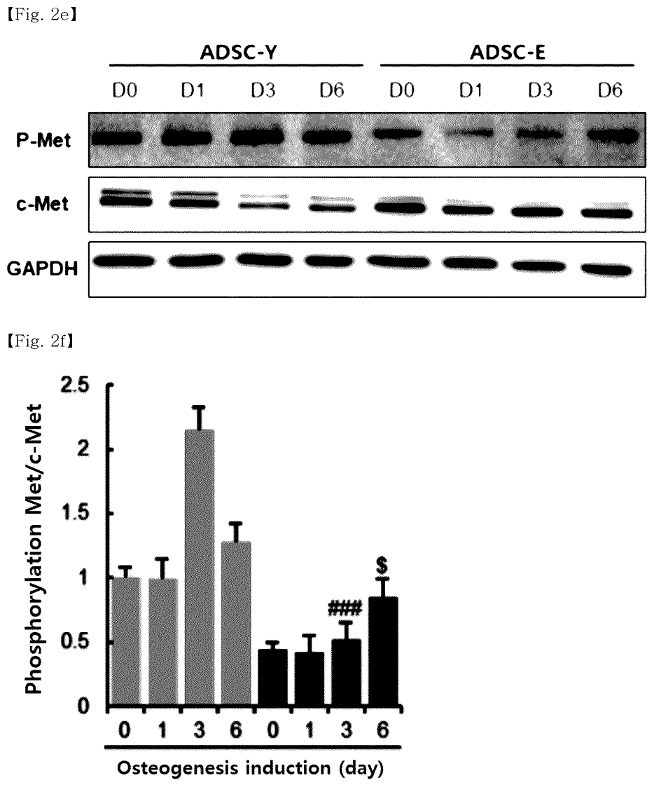

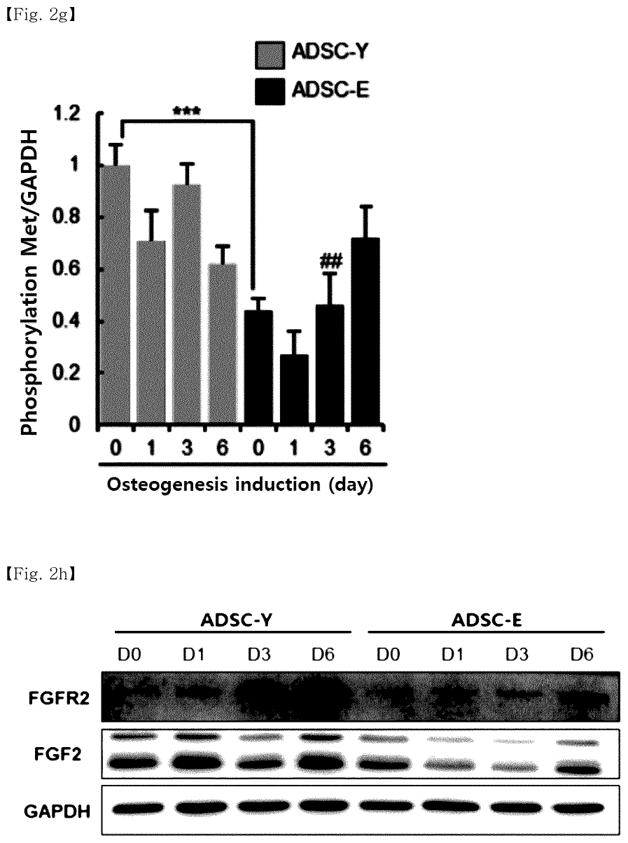

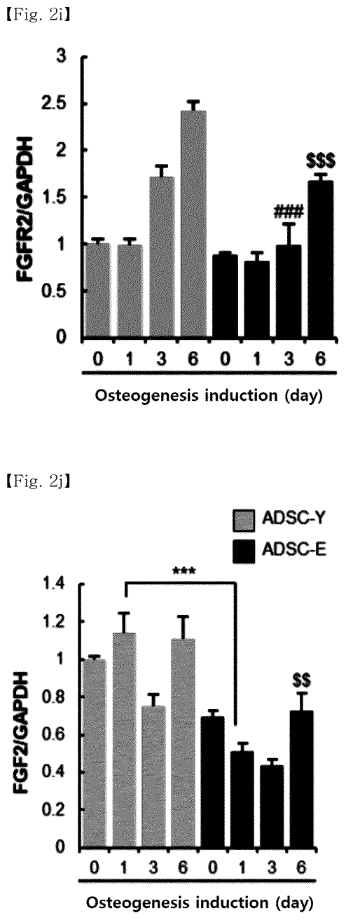

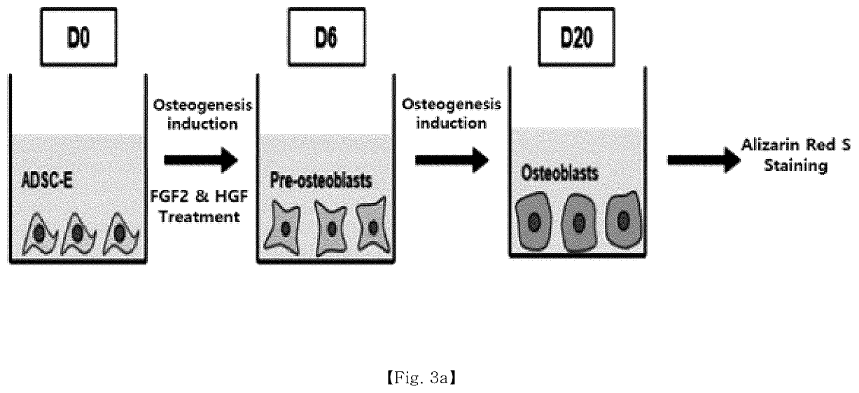

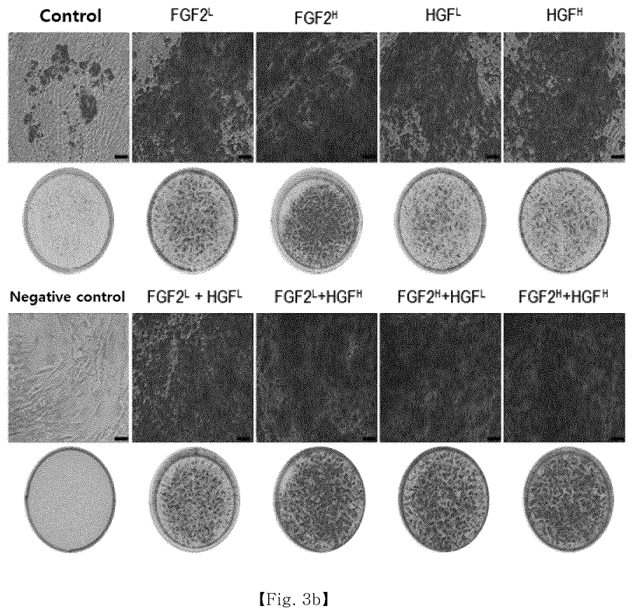

As a result, the concentration of BMP-2 in ADSC-E was increased to a similar level to in ADSC-Y by osteogenesis induction/stimulation (FIG. 2A). In addition, the level of TGF-Beta1 was maintained in a similar pattern in both ADSC-Y and ADSC-E under osteogenic conditions (FIG. 2B), and VEGF secretion increased stepwise in ADSC-Y, whereas showed almost no change for 6 days in ADSC-E (FIG. 2C). In addition, the level of HGF in ADSC-Y was consistently increased after osteogenesis induction, whereas the level thereof was low that was undetectable in ADSC-E (FIG. 2D).

### Example 6. Analysis of Expression Patterns of Osteogenic Factors According To Osteogenesis Induction

Since HGF bound to a receptor c-Met to be autophosphorylated, thereby inducing various signaling pathways, to confirm the expression patterns of osteogenic factors P-Met and C-Met according to the induction of osteogenesis, Western blot analysis was performed using primary antibodies against C-Met, P-Met, FGF2 (Cell Signaling Technology, Danvers, MA, USA), fibroblast growth factor receptor 2 (FGFR2), and GAPDH (Abcam, Cambridge, UK) in ADSC-Y and ADSC-E, respectively, by the method of Example 3.

As a result, ADSC-Y, which actively secreted HGF, showed a significantly higher level of C-Met phosphorylation than ADSC-E (FIGS. 2E to 2G). In addition, it was shown that the expression of FGF2 and FGF2R was significantly higher maintained in ADSC-Y than in ADSC-E (FIGS. 2H to 2J).

Considering a difference in the paracrine potential between ADSC-Y and

ADSC-E in the early stage of osteogenesis induction confirmed in Examples, it is not expected that BMP-2 or TFG-Beta will be directly associated with the loss of osteogenic function of ADSC-E, which is because the difference between ADSC-E and ADSC-Y is not significant. Therefore, it can be inferred that the deficiency of HGF, FGF2, or VEGF directly affects the osteogenic function of damaged ADSC-E.

### Example 7. Confirmation of Promotion of Osteogenic Differentiation of ADSCs by FGF2 and HGF

To determine whether supplementation of FGF2 and/or HGF in ADSC-E with low osteogenic potential could restore the osteogenic function, ADSC-E was primed with FGF2 and/or HGF using the same process as in FIG. 3A, and its osteogenic function was confirmed by Alizarin Red S staining. Specifically, to clarify the optimal time for differentiation stimulation of ADSC-E by FGF2 and/or HGF, the culture medium of ADSC-E was replaced with differentiation media (Stempro osteogenesis differentiation media) containing FGF2 (R&D systems, Minneapolis, MN, USA) (1 or 5 ng/ml), HGF (R&D systems, Minneapolis, MN, USA) (10 or 50 ng/mL), or FGF2+HGF, and while osteogenesis was induced for 20 days, the osteogenic function was confirmed on days 3 and 6 (FIG. 3A). At this time, during the differentiation period of 20 days, treatment with HGF or FGF was performed for initial 3 or 6 days, and a basic osteogenic differentiation medium without FGF/HGF was treated for the remaining 17 or 14 days.

As a result, it was shown that the osteogenic function of ADSC-E was significantly enhanced by FGF2 or HGF treatment for 6 days (FIG. 3B). In particular, the effect of FGF-2 was hardly observed in ADSC-Y, but was significantly shown in ADSC-E, which indicated the importance of FGF-2 supplementation for the osteogenic function. In addition, when treated with a combination of FGF2 and HGF, the osteogenic potential of ADSC-E was significantly restored to a level similar to that of ADSC-Y (FIG. 3). To determine the exact effect of each condition, Alizarin Red S was quantified, and as a result, a particularly significant improvement was observed when FGF2 and HGF were combined and treated, compared to when FGF2 or HFG was treated alone (FIG. 3C). Through this, the optimal treatment concentrations of FGF2 or HGF were determined as 5 ng/mL FGF2 and 50 ng/mL HGF, respectively, and were used in subsequent experiments.

These results confirmed that supplementation of FGF2 and/or HGF for 6 days of initial differentiation could promote the osteogenic capacity of ADSC-E.

### Example 8. Confirmation of Osteogenesis-Enhancing Mechanism of FGF2 and HGF

### 8-1. Confirmation of Changes in Expression of Early Osteogenic Markers

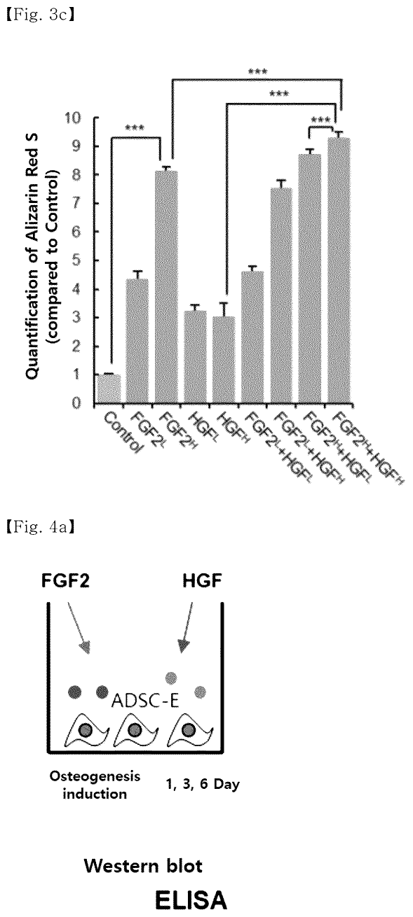

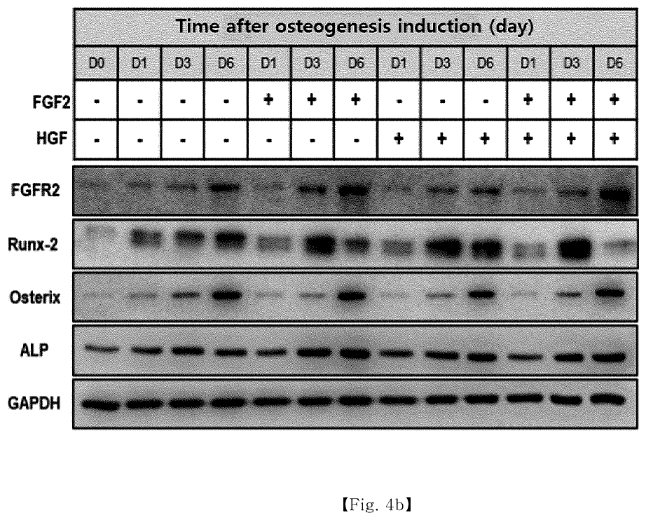

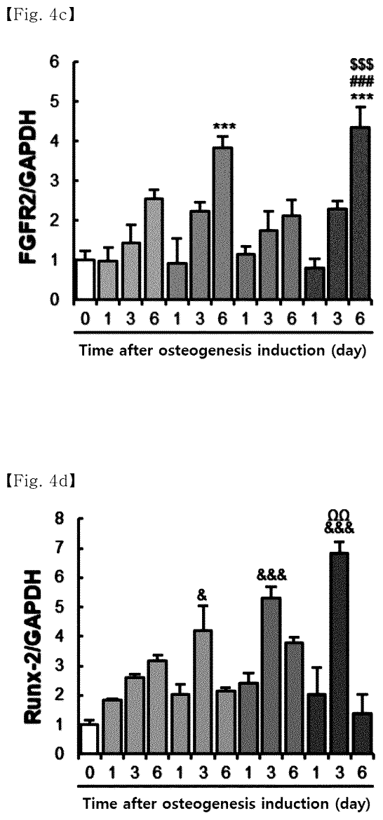

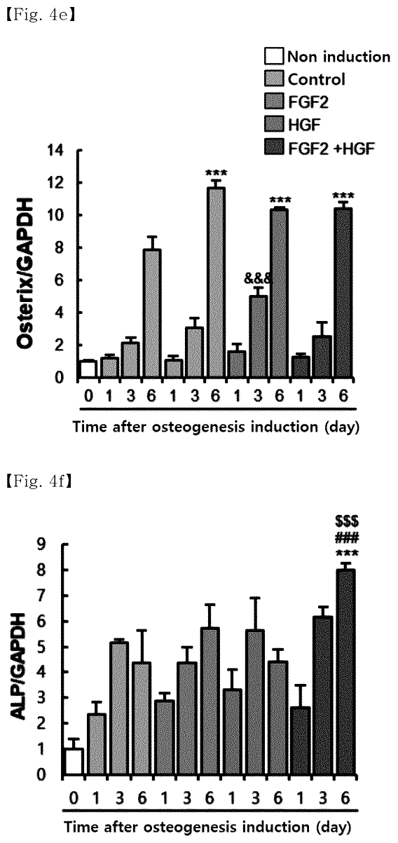

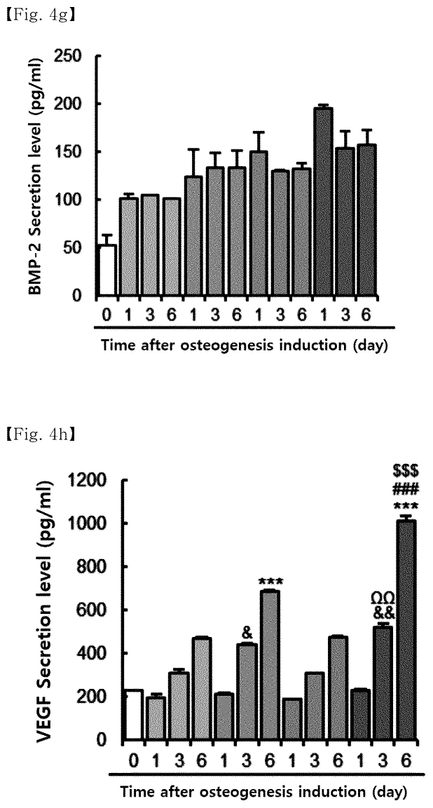

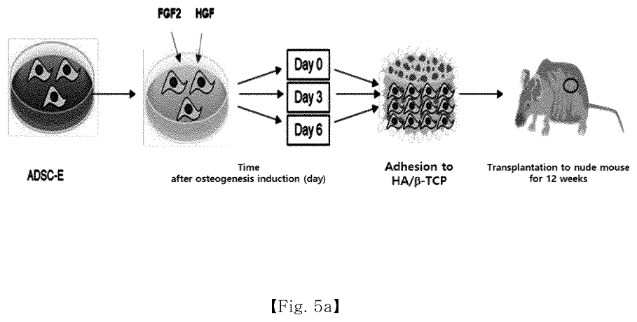

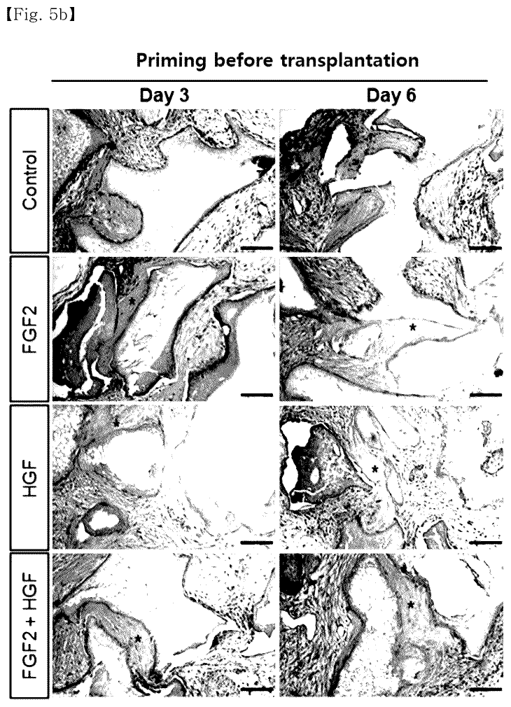

To determine which protein expression was changed by FGF2 and HGF treatment to enhance osteogenic cell differentiation of ADSC-E, ADSC-E was co-treated with FGF2 and/or HGF during osteogenesis induction (untreated group: control group) and then the expression changes of early osteogenic markers FGFR2, Runx-2, Osterix, and ALP of ADSC-E were confirmed by Western blot analysis on days 1, 3, and 6 (FIG. 4A).

As a result, under osteogenic conditions, the expression of FGFR2 increased in a time-dependent manner, which was significantly shown under FGF2 or FGF2+HGF conditions (FIGS. 4B and 4C). In addition, Runx-2 showed a time-dependent increase in the untreated group, but the Runx-2 expression was promoted by FGF2 and/or HGF treatment, its peak was shifted to day 3, and the Runx-2 expression level was highest when treated with the combination of FGF2 and HGF (FIGS. 4B and 4D). In addition, Osterix and ALP expressions were found to be slightly changed upon FGF2 and/or HGF treatment compared to the control group (FIGS. 4B, 4E, and 4F).

Based on the protein expression profiles, it was predicted that FGF2 and/or HGF would introduce ADSC-E into an immature pre-osteoblast phase when compared to the ADSC-E control group untreated with FGF2 and/or HGF.

### 8-2. Confirmation of Changes in Secretion of Secretory Factors

To determine which protein expression was changed by FGF2 and HGF treatment to enhance osteogenic cell differentiation of ADSC-E, ADSC-E was co-treated with FGF2 and/or HGF during osteogenesis induction (untreated group: control group) and then on days 1, 3, and 6, among secretory factors with different basal levels in ADSC-E and ADSC-Y, the secretion levels of BMP-2 and VEGF were evaluated by ELISA.

As a result, when FGF2 or HGF was treated alone, the secretion level of BMP-2 was not significant between the groups, but when FGF2 and HGF were treated in combination, the highest concentration was observed from day 1 and was maintained at a significantly higher level than the control group (FIG. 4G). In addition, VEGF secretion was increased by osteogenesis induction, and its concentration was not affected at all by FGF2 or HGF. However, when FGF2 and HGF were combined, VEGF secretion from ADSC-E was significantly increased (FIG. 4H).

### Example 9. Confirmation of Enhanced Osteogenic Capacity of ADSCs by in vivo FGF2 and HGF

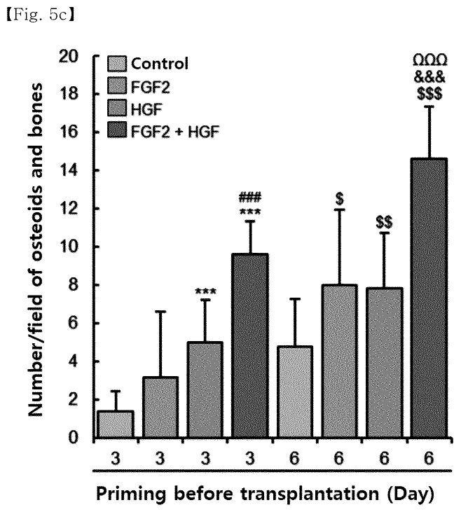

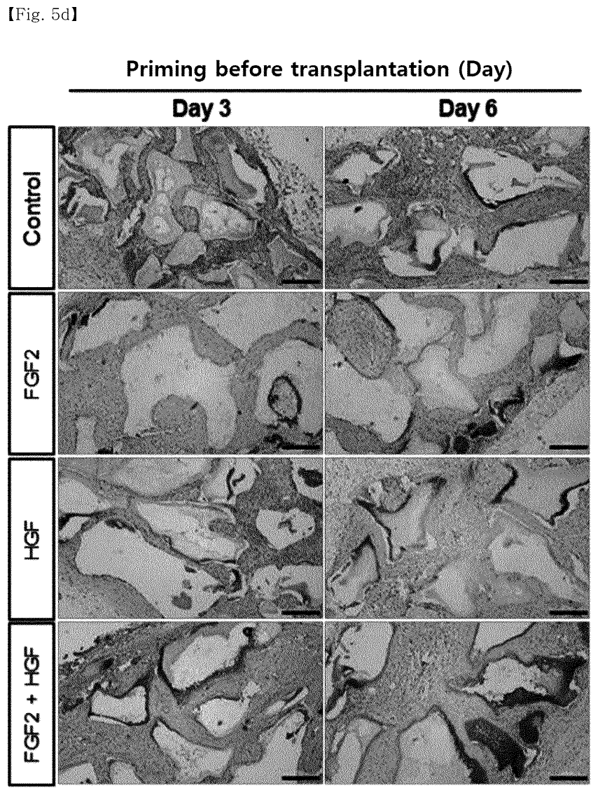

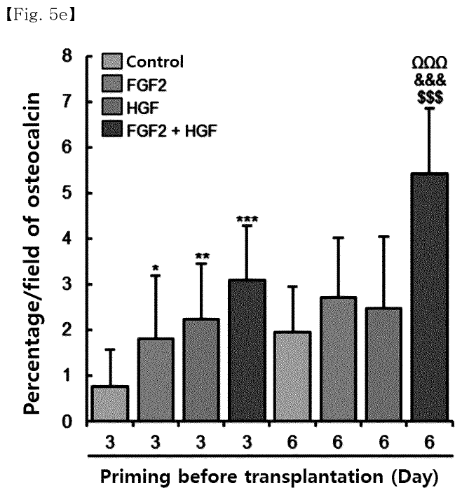

To evaluate the osteogenic capacity of ADSCs treated with FGF2 and/or HGF in vivo, ADSCs were treated with FGF2 and/or HGF under osteogenesis induction, and then synthesized by osteoblasts, and stained with osteocalcin, an important bone formation marker to confirm the osteogenic capacity. Specifically, ADSCs were treated together with FGF2, HGF, and FGF2+HGF for 3 and 6 days, respectively, during osteogenesis induction, and then 2×10⁶ ADSCs were mixed with 40 mg of hydroxyapatite/beta-tricalcium phosphate (HA/B-TCP) ceramic powder (Biomatlante, Vigneux-de-bretagne, France). The ADSC-HA/B-TCP mixture was incubated at 37° C. for 2 hours and then subcutaneously transplanted into the back of 6-week-old male Balb/c nude mice (20 to 22 g) (FIG. 5A). After 12 weeks, the implants were recovered and fixed in 3.7% formaldehyde. The samples were decalcified with 0.2 M EDTA (pH 7.2 to 7.4) for 2 weeks and embedded in paraffin. The paraffin-embedded samples were sectioned at a 5-μm thickness, deparaffinized and hydrated, and then stained with hematoxylin and eosin (H&E). To detect transplanted human ADSCs, the samples were treated with antibodies against human osteocalcin and incubated with a biotin-conjugated secondary antibody. The enzyme-substrate reaction was performed with an ABC reagent solution. Stained sections were visualized with Nova RED (Vector Laboratories, Burlingame, CA, USA), and counterstaining was completed with hematoxylin.

H&E staining results showed that osteoids and newly formed bones were observed, and the FGF2 and/or HGF-treated groups showed enhanced bone formation compared to the control group (untreated group with FGF2 and/or HGF) (FIGS. 5B and 5C). In particular, cells in the group treated with a combination of FGF2 and HGF showed significantly enhanced bone formation on both days 3 and 6 of osteogenesis induction compared to cells in the group treated with FGF2 or HGF alone, and the highest osteogenic capacity was observed in ADSCs treated with a combination of FGF2 and HGF for 6 days (FIGS. 5B and 5C). In addition, as a result of confirming immunohistochemically the expression of human-specific osteocalcin, a small area positive for osteocalcin in the control group was observed, which was significantly increased by FGF2 and/or HGF priming (FIGS. 5D and 5E). In particular, the area stained with osteocalcin was most significantly increased in the group treated with a combination of FGF2 and HGF for 6 days (FIGS. 5D and 5E), which was confirmed that the combined treatment of FGF2 and HGF most significantly improved the osteogenic function of ADSC-E, and promoted differentiation into osteoblasts in vivo.

Through Examples, it was confirmed that the initial priming by the combination of FGF2 and HGF was necessary for improving the osteogenic function of ADSC-E both in vitro and in vivo.

1. A method for promoting differentiation of stem cells, comprising treating stem cells with a composition comprising a fibroblast growth factor (FGF) 2 or a hepatocyte growth factor (HGF) as an active ingredient.

2. The method of claim 1, wherein the composition comprises FGF2 and HGF.

3. The method of claim 1, wherein FGF2 is included at a concentration of 0.5 to 10 ng/ml.

4. The method of claim 1, wherein HGF is included at a concentration of 5 to 100 ng/mL.

5. The method of claim 1, wherein the stem cells are adult stem cells.

6. The method of claim 5, wherein the adult stem cells are derived from at least one selected from the group consisting of bone marrow, blood, brain, skin, fat, umbilical cord blood, and umbilical cord Wharton's jelly.

7. The method of claim 1, wherein the composition promotes the differentiation of the stem cells into osteocytes.

8. The method of claim 1, wherein the method promotes differentiation of adipose-derived stem cells (ADSCs) into osteoblasts.

9. The method of claim 1, wherein the stem cells are adipose-derived stem cells derived from elderly donors aged 50 to 80 years.

10. A method for inducing osteogenic differentiation of stem cells, comprising treating stem cells with a composition comprising FGF2 or HGF as an active ingredient.

11. The method of claim 10, wherein the method increases a differentiation induction effect when inducing osteocyte differentiation.

12. The method of claim 11, wherein the osteocytes are osteoblasts.

13. A method for preventing or treating bone disease, comprising administering to a subject in need thereof a composition comprising FGF2 or HGF as an active ingredient.

14. The method for preventing or treating bone disease of claim 13, wherein the bone disease is at least one selected from the group consisting of arthritis, bone defect disease, osteoporosis, osteopenia, osteolytic metastasis, senile kyphosis, and Paget disease.

15. (canceled)

16. A method for enhancing osteogenic differentiation potential of stem cells, comprising:
(a) isolating adipose-derived adult stem cells extracted from adipocytes isolated from a subject; and
(b) treating the stem cells with the composition of claim 10.

17. The method for enhancing osteogenic differentiation potential of stem cells of claim 16, wherein step (b) is replacing a medium with a differentiation medium containing FGF2 or HGF.

18. The method for enhancing osteogenic differentiation potential of stem cells of claim 16, wherein the differentiation potential of adipose-derived stem cells into osteoblasts is enhanced.

19-21. (canceled)

22. A method for treating bone disease, comprising transplanting the FGF2 or HGF of claim 13 into a subject suffering from bone disease.

23. A method for treating bone disease, comprising transplanting stem cells treated with the FGF2 or HGF of claim 13 into a subject suffering from bone disease.
